(19) United States
(12) Patent Application Publication
(10) Pub. No.: US 2025/0132057 A1
(43) Pub. Date: Apr. 24, 2025

(54) INFECTIOUS DISEASE INFECTION PREDICTION METHOD, APPARATUS, AND STORAGE MEDIUM BASED ON MACRO-MICROGRAPH FUSION

(71) Applicant: ZHEJIANG LAB, Hangzhou (CN)

(72) Inventors: Zenghui XU, Hangzhou (CN); Ji ZHANG, Hangzhou (CN); Yu ZHANG, Hangzhou (CN); Ting YU, Hangzhou (CN); Jin ZHAO, Hangzhou (CN); Linlin HOU, Hangzhou (CN); Zhan ZHANG, Hangzhou (CN)

(21) Appl. No.: 18/600,800

(22) Filed: Mar. 11, 2024

Related U.S. Application Data
(63) Continuation of application No. PCT/CN2023/135675, filed on Nov. 30, 2023.

(30) Foreign Application Priority Data
Oct. 20, 2023 (CN) 202311366722.4

(51) Int. Cl. G16H 50/80

(52) U.S. Cl. CPC G16H 50/80

(57) ABSTRACT

An infectious disease infection prediction method, an apparatus, and a storage medium based on macro-micrograph fusion are provided. The method includes: acquiring macrographs of a plurality of first regions and micrographs of second regions within a set period; inputting the macroscopic graphs and the microscopic graphs into two graph convolutional neural networks to obtain two hidden layer vectors respectively, and fusing the two hidden layer vectors to obtain fusion hidden layer information of the first regions; performing a time sequence calculation of the fusion hidden layer information to obtain time sequence hidden layer information of the first regions; inputting the time series hidden layer information into two prediction networks to obtain two prediction results, respectively, and performing fusion calculation of the two prediction results to obtain a final prediction result of infectious diseases in the first regions.

### CROSS-REFERENCE TO RELATED APPLICATIONS

This application is a continuation of international patent application No. PCT/CN2023/135675, filed on Nov. 30, 2023, which claims priority to Chinese patent applications No. 202311366722.4, filed on Oct. 20, 2023, titled “INFECTIOUS DISEASE INFECTION PREDICTION METHOD, APPARATUS, AND STORAGE MEDIUM BASED ON MACRO-MICROGRAPH FUSION”. The contents of the above applications are hereby incorporated by reference.

### TECHNICAL FIELD

The present disclosure generally relates to the field of machine learning, and in particular, to an infectious disease infection prediction method, an apparatus, and a storage medium based on macro-micrograph fusion.

### BACKGROUND

Accurate prediction of the number of future infections of infectious diseases has been an important issue in the field of infectious disease research. A conventional prediction method is a method based on a mechanistic model SIR (Susceptible, Infective, Recovered), but this method requires to set many parameters artificially based on experience, with great uncertainty and unsatisfactory prediction effect.

Some neural network and machine learning methods have also been applied to infectious disease infection prediction, but these methods use the number of infections of infectious diseases to perform a simple prediction of the number of people, which cannot dig out a hidden internal relationship among infectious disease data, prediction accuracy of these methods is not high, and at the same time, prediction results cannot be interpreted.

### SUMMARY

According to various embodiments of the present disclosure, an infectious disease infection prediction method, an apparatus, and a storage medium based on macro-micrograph fusion are provided.

In a first aspect, an infectious disease infection prediction method based on macro-micrograph fusion is provided in the present disclosure, including:
acquiring macro infection data of a plurality of first regions within a set period to generate macrographs, and acquiring micro infection data of a plurality of second regions within the same set period to generate micrographs corresponding to the plurality of first regions, wherein each of the plurality of first regions includes the plurality of second regions;
inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions;
performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively; and
performing a fusion calculation of the first result and the second result to obtain an infectious disease prediction result of the plurality of first regions.

In an embodiment, the macro infection data includes macro personnel data and macro geographic data of the plurality of first regions, and acquiring macro infection data of the plurality of first regions within the set period to generate the macrographs further includes:
taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs, wherein the macro personnel data includes: a total population, a population density, the number of infected persons, and the number of recovered persons in each of the plurality of first regions within the set period.

In an embodiment, determining first connecting edges of the first nodes based on the macro geographic data of the plurality of first regions further includes:
determining a first connecting edge probability between the first nodes based on the macro geographic data of the plurality of first regions, connecting two first nodes between which the first connecting edge probability is greater than a set threshold value, and obtaining the first connecting edges, wherein the macro geographic data includes: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

In an embodiment, the micro infection data includes micro personnel data and micro geographic data of the plurality of second regions, and acquiring micro infection data of the plurality of second regions within the same set period to generate micrographs corresponding to the plurality of first regions includes:
taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph, wherein the micro personnel data includes: a total population, a population density, and the number of hospitals in each of the plurality of second regions within the set period.

In an embodiment, determining second connecting edges of the second nodes based on the micro geographic data of the plurality of second regions further includes:
determining a second connecting edge probability between the second nodes based on the micro geographic data of the plurality of second regions, connecting two second nodes between which the second connecting edge probability is greater than a set threshold, and obtaining the second connecting edges, wherein the micro geographic data includes: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

In an embodiment, performing the time sequence calculation of the fusion hidden layer information to obtain time sequence hidden layer information of the plurality of first regions further includes:
acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

In an embodiment, the first result includes a first prediction number of infected persons and a first prediction number of recovered persons, the second result includes a second prediction number of infected persons and a second prediction number of recovered persons, the second prediction network includes a parameter prediction network and an infection prediction network, and inputting the time sequence hidden layer information into the first prediction network and the second prediction network to obtain the corresponding first result and the corresponding second result respectively further includes:
inputting the time sequence hidden layer information into the first prediction network, and outputting the first prediction number of infected persons and the first prediction number of recovered persons in the plurality of first regions; and
inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

In an embodiment, performing the fusion calculation of the first result and the second result to obtain the infectious disease prediction result of the plurality of first regions further includes:
performing a fusion calculation of the first prediction number of infected persons and the second prediction number of infected persons to obtain a prediction result of the number of infected persons in the plurality of first regions; and
performing a fusion calculation of the first prediction number of recovered persons and the second prediction number of recovered persons to obtain a prediction result of the number of recovered persons in the plurality of first regions.

In a second aspect, an infectious disease infection prediction apparatus based on macro-micrograph fusion is provided in the present disclosure, including a graph calculation processing module, a spatial fusion calculation module, a time fusion calculation module, and a fusion result module;
the graph calculation processing module is configured for acquiring macro infection data of a plurality of first regions within a set period to generate macrographs, and acquiring micro infection data of a plurality of second regions within the same set period to generate micrographs corresponding to the plurality of first regions; each of the plurality of first regions includes the plurality of second regions;
the spatial fusion calculation module is configured for inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions;
the time fusion calculation module is configured for performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively; and
the fusion result module is configured for performing a fusion calculation of the first result and the second result to obtain an infectious disease prediction result of the plurality of first regions.

In a third aspect, a computer-readable storage medium is provided in the present disclosure. The computer-readable storage medium stores a computer program, and the computer program is executed by a processor to implement the method in the first aspect.

Details of one or more embodiments of the present disclosure are set forth in the following accompanying drawings and descriptions. Other features, objectives, and advantages of the present disclosure become obvious with reference to the specification, the accompanying drawings, and the claims.

### BRIEF DESCRIPTION OF THE DRAWINGS

In order to more clearly illustrate the technical solutions in the embodiments of the present disclosure or the related technology, the accompanying drawings to be used in the description of the embodiments or the related technology will be briefly introduced below, and it will be obvious that the accompanying drawings in the following description are only some of the embodiments of the present disclosure, and that, for one skilled in the art, other accompanying drawings can be obtained based on these accompanying drawings without putting in creative labor.

FIG. 1 is a diagram of an application environment of an infectious disease infection prediction method based on macro-micrograph fusion in an embodiment.

FIG. 2 is a flowchart of an infectious disease infection prediction method based on macro-micrograph fusion in an embodiment.

FIG. 3 is a schematic diagram of constructing a macrograph in an embodiment.

FIG. 4 is a schematic diagram of constructing a micrograph in an embodiment.

FIG. 5 is a block diagram of a structure of an infectious disease infection prediction apparatus based on macro-micrograph fusion in an embodiment.

FIG. 6 is an internal structure diagram of a FPGA device in an embodiment.

### DETAILED DESCRIPTION OF THE EMBODIMENT

The technical solutions in the embodiments of the present disclosure will be described clearly and completely in the following in conjunction with the accompanying drawings in the embodiments of the present disclosure, and it is obvious that the described embodiments are only a part of the embodiments of the present disclosure, but not all of the embodiments. Based on the embodiments in the present disclosure, all other embodiments obtained by one skilled in the art without making creative labor fall within the scope of protection of the present disclosure.

Unless defined otherwise, technical terms or scientific terms involved in the present disclosure have the same meanings as would generally understood by one skilled in the technical field of the present disclosure. In the present disclosure, “a”, “an”, “one”, “the”, and other similar words do not indicate a quantitative limitation, which may be singular or plural. The terms such as “comprise”, “include”, “have”, and any variants thereof involved in the present disclosure are intended to cover a non-exclusive inclusion. For example, processes, methods, systems, products, or devices including a series of steps or modules (units) are not limited to these steps or modules (units) listed, and may include other steps or modules (units) not listed, or may include other steps or modules (units) inherent to these processes, methods, systems, products, or devices. Words such as “join”, “connect”, “couple”, and the like involved in the present disclosure are not limited to physical or mechanical connections, and may include electrical connections, whether direct or indirect. “A plurality of” involved in the present disclosure means two or more. The term “and/or” describes an association relationship between associated objects and represents that three relationships may exist. For example, A and/or B may represent the following three cases: only A exists, both A and B exist, and only B exists. Generally, a character “/” indicates an “or” relationship between the associated objects. The terms “first”, “second”, “third”, and the like involved in the present disclosure are only intended to distinguish similar objects and do not represent specific ordering of the objects.

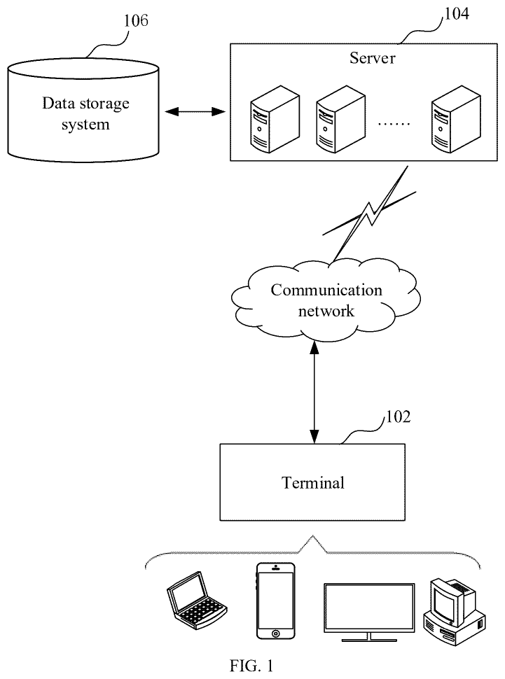

An infectious disease infection prediction method based on macro-micrograph fusion provided in an embodiment of the present disclosure may be applied in an application environment as shown in FIG. 1. A terminal 102 may be in communication with a server 104 by a network. A data storage system 106 may store data to be processed by the server 104. The data storage system 106 may be integrated in the server 104 or may be placed on a cloud or other network server.

The server 104 may acquire macro infection data of a plurality of first regions within a set period to generate macrographs, and acquire micro infection data of a plurality of second regions within the same set period to generate micrographs corresponding to the plurality of first regions. Each of the plurality of first regions includes the plurality of second regions. The server 104 may further input the macrographs into a first graph convolutional neural network to obtain a first hidden layer vector, input the micrographs into a second graph convolutional neural network to obtain a second hidden layer vector, and perform a fusion calculation of the first hidden layer vector and the second hidden layer vector to obtain fusion hidden layer information of the plurality of first regions; perform a time sequence calculation of the fusion hidden layer information to obtain time sequence hidden layer information of the plurality of first regions, and input the time sequence hidden layer information into a first prediction network and a second prediction network to obtain a corresponding first result and a corresponding second result, respectively; and perform a fusion calculation of the first result and the second result to obtain an infectious disease prediction result of the plurality of first regions.

The terminal 102 may be, but is not limited to, a variety of personal computers, laptops, smartphones, tablets, and the like. The server 104 may be realized with a stand-alone server or a server cluster including a plurality of servers.

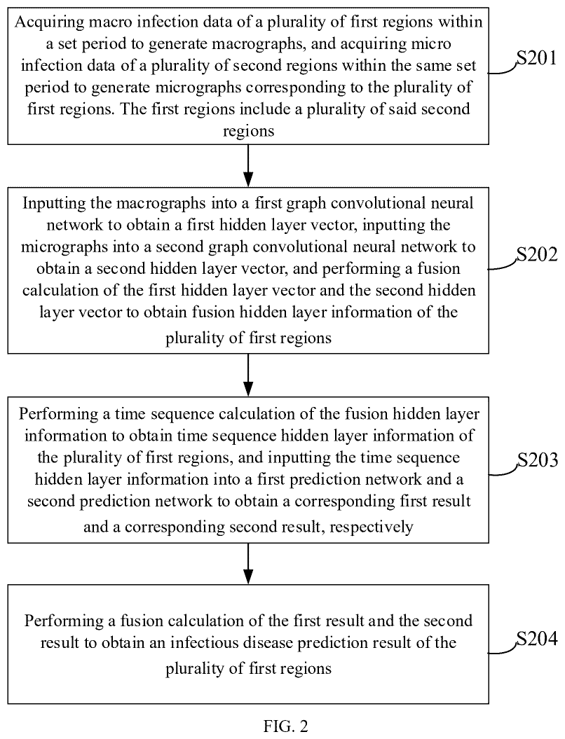

In an embodiment, referring to FIG. 2, an infectious disease infection prediction method based on macro-micrograph fusion is provided, as illustrated by an example of the method being applied to the server in FIG. 1, including following step 201 to step 204.

Step 201 includes acquiring macro infection data of a plurality of first regions within a set period to generate macrographs, and acquiring micro infection data of a plurality of second regions within the same set period to generate micrographs corresponding to the plurality of first regions. The first regions include a plurality of said second regions.

The macro infection data may include macro personnel data and macro geographic data of the plurality of first regions. The micro infection data may include micro personnel data and micro geographic data of the plurality of second regions.

Specifically, macro personnel data and macro geographic data of the plurality of first regions in spread of an infectious disease, and micro personnel data and micro geographic data of the plurality of second regions in each first region may be acquired to construct the macrographs and the micrographs of the plurality of the first regions. Multiple aspects of infectious disease factors may be incorporated to increase a size of a processing dataset for infectious disease prediction, resulting in a more accurate prediction result.

Step 202 include inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions.

Specifically, an infectious disease prediction model may be constructed, the infectious disease prediction model may include the first graph convolutional neural network and a second graph convolutional neural network. The macrographs may be input into the first graph convolutional neural network to obtain the first hidden layer vector of the macrographs, the micrographs may be input into the second graph convolutional neural network to obtain the second hidden layer vector, and the first hidden layer vector may be fused with the second hidden layer vector to obtain the fusion hidden layer information of the plurality of first regions. A fused macro-micrograph hidden layer vectors may be utilized to comprehensively mine an association between various types of data and a predicted result of the infectious disease.

Step 203 includes performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively.

Specifically, the infectious disease prediction model may further include a recurrent control network, a first prediction network, and a second prediction network. The second prediction network may include an infectious disease mechanism model. The fusion hidden layer information may be input into the recurrent control network for time sequence calculation to obtain the time sequence hidden layer information of the plurality of first regions, and then the time sequence hidden layer information may be input into the first prediction network and the second prediction network to obtain two prediction results of the infectious disease, respectively.

Step 204 includes performing a fusion calculation of the first result and the second result to obtain an infectious disease prediction result of the plurality of first regions.

Specifically, the fusion calculation of the two prediction results of the infectious disease may be performed to obtain the infectious disease prediction result of a corresponding first region. The fusion of spatial-temporal deep learning and the infectious disease mechanism model may improve interpretability of a final output infectious disease prediction result.

In the above infectious disease infection prediction method based on macro-micrograph fusion, the macro infection data of the plurality of first regions within the set period may be acquired to generate the macrographs, and the micro infection data of the plurality of second regions within the same set period may be acquired to generate the micrographs corresponding to the plurality of first regions; each of the plurality of first regions may include the plurality of second regions; the macrographs may be input into the first graph convolutional neural network to obtain the first hidden layer vector, the micrographs may be input into the second graph convolutional neural network to obtain the second hidden layer vector, and the fusion calculation of the first hidden layer vector and the second hidden layer vector may be performed to obtain the fusion hidden layer information of the plurality of first regions; the time sequence calculation of the fusion hidden layer information may be performed to obtain the time sequence hidden layer information of the plurality of first regions, and the time sequence hidden layer information may be input into the first prediction network and the second prediction network to obtain the corresponding first result and the corresponding second result, respectively; and the fusion calculation of the first result and the second result may be performed to obtain the infectious disease prediction result of the plurality of first regions. The method may realize infectious disease prediction by macro-micrograph fusion and improve the interpretability of the prediction result and accuracy and efficiency of the infectious disease prediction.

In an embodiment, at the step 201, acquiring macro infection data of the plurality of first regions within the set period to generate the macrographs may further include: taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs.

The macro personnel data may include: a total population, a population density, the number of infected persons, and the number of recovered persons in each of the plurality of first regions within the set period.

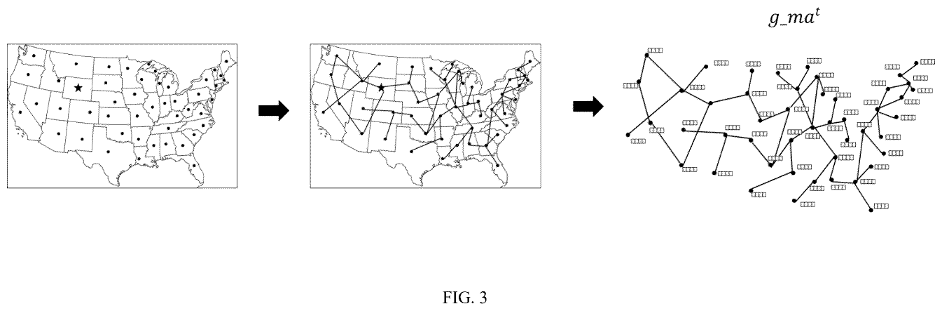

Specifically, within the set period denoted as t, referring to FIG. 3, the macro infection data of the plurality of first regions may be abstracted into a macrograph denoted as g_ma^{t}=(Vₛ^{t}, Eₛ^{t}). Vₛ^{t} represents the first nodes corresponding to the plurality of first regions, and Est represents a combination of the first connecting edges of the first nodes. The total population, the population density, the number of infected persons, and the number of recovered persons in each of the plurality of first regions within the set period/may be taken as the node features denoted as s of a corresponding first node Vₛ^{t}.

Determining first connecting edges of the first nodes based on the macro geographic data of the plurality of first regions may further include: determining a first connecting edge probability between the first nodes based on the macrogeographic data of the plurality of first regions, connecting two first nodes between which the first connecting edge probability is greater than a set threshold, and obtaining the first connecting edges.

The macro geographic data may include: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

Specifically, within the set period t, the number of infected persons flowing from a first region denoted as i to another first region denoted as j is denoted as Sᵣₑₘ_{_{i,j}}^{t}, the total number of infected persons flowing out from the first region i is denoted as Σ_{kEN(i)}Sᵣₑₘ_{_{i,k}}^{t}, the historical number of infected persons in the first region i is denoted as observerᵢ^{t}, the historical number of infected persons in the another first region j is denoted as observeᵢ^{t}, and the geographic index between the first region i and the another first region j is denoted as ∈. The first connecting edge probability between the first nodes denoted ase_s_{i,j}^{t}, may be calculated by a following formula:

$${e\_s}_{i , j}^{t} = a * \frac{{s}_{{rem}_{i , j}}^{t}}{{\sum }_{k ∈ N ( i )} {s}_{{rem}_{i , k}}^{t}} + b * simil ( {observe}_{i}^{t} , {observe}_{j}^{t} ) + ∈ ,$$

a and b represent manually set weight parameters, simil represents a cosine similarity calculation function, N represents the total number of neighboring regions to the first region i, k represents the other first regions to which the infected people in the first region i flow, which is configured for calculating infection similarity between two first regions, and a formula for calculating the geographic index E between the first region i and the another first region j is:

$$∈ = \{ \begin{matrix}0 & ( i and j are not geographically adjacent ) \\ \frac{1}{N} & ( \begin{matrix}i and j are geographically adjacent , and \\ N is the total number of adjacent regions of i\end{matrix} )\end{matrix} .$$

In the present embodiment, the first connecting edge probability of the macrographs may be calculated by the macro geographic data of the plurality of first regions, and two first regions with the first connecting edge probability greater than the set threshold may be connected to realize construction of the macrographs. Population flow, the total population, geographic location, the population density, and the number of infected persons may be combined to improve comprehensiveness of the macrographs, thereby enhancing reliability of the prediction result of the infectious disease.

In an embodiment, at the step 201, acquiring micro infection data of the plurality of second regions within the same set period to generate micrographs corresponding to the plurality of first regions may further include: taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph.

The micro personnel data may include: a total population, a population density, and the number of hospitals in each of the plurality of second regions within the set period.

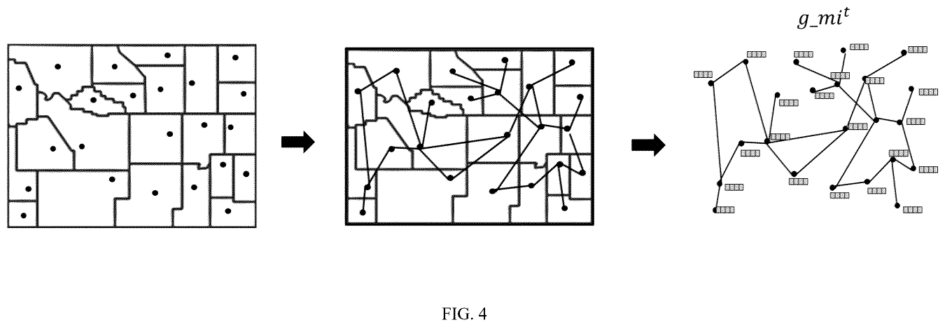

Specifically, within the same set period t, referring to FIG. 4, the micro infection data of the plurality of second regions may be abstracted into a micrograph of a corresponding first region denoted as g_mi^{t}=(V_{c}^{t}, E_{c}^{t}). V_{c}^{t} represents the second nodes corresponding to the plurality of second regions, and Fc represents a combination of the second connecting edges of the second nodes. The total population, the population density, and the number of hospitals in each of the plurality of second regions within the set period/may be taken as the node features denoted as c of a corresponding second node V_{c}^{t}.

Determining second connecting edges of the second nodes based on the micro geographic data of the plurality of second regions may further include: determining a second connecting edge probability between the second nodes based on the micro geographic data of the plurality of second regions, connecting two second nodes between which the second connecting edge probability is greater than a set threshold, and obtaining the second connecting edges.

The micro geographic data may include: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

Specifically, within the same set period t, the number of infected persons flowing from a second region denoted as m to another second region denoted as n is denoted as Cᵣₑₘ_{_{m,n}}^{t}, the total number of infected persons flowing out from the second region m is denoted as Σ_{K∈N(m)}Sᵣₑₘ_{_{m,k}}^{t}, and the geographic index between the second region m and the another second region n is denoted as ∈′. The second connecting edge probability between the second nodes denoted as e_c_{m,n}^{t} may be calculated by a following formula:

$${e\_c}_{m , n}^{t} = {a}^{′} * \frac{{c}_{{rem}_{m , n}}^{t}}{{\sum }_{k ∈ N ( m )} {c}_{{rem}_{m , k}}^{t}} + {∈}^{′} ,$$

a′ represents a manually set weight parameter, and a formula for calculating the geographic index ∈′ between the second region m and the another second region n is:

$${∈}^{′} = \{ \begin{matrix}0 & ( m and n are not geographically adjacent ) \\ \frac{1}{N} & ( \begin{matrix}m and n are geographically adjacent , and \\ N is the total number of adjacent regions of m\end{matrix} )\end{matrix} .$$

In the present embodiment, the second connecting edge probability of the micrographs may be calculated by the micro geographic data of the plurality of second regions, and two second regions with the second connecting edge probability greater than the set threshold may be connected to realize construction of the micrographs. Population flow, the total population, geographic location, the population density, the number of infected persons, and the number of hospitals may be combined to improve comprehensiveness of the micrographs, thereby enhancing the reliability of the prediction result of the infectious disease.

In an embodiment, at the step 202, performing a fusion calculation of the first hidden layer vector and the second hidden layer vector to obtain fusion hidden layer information of the plurality of first regions may further includes: performing a fusion calculation of the first hidden layer vector denoted as

$${h}_{{m \_ i}_{i}}^{t}$$

and the second hidden layer vector denoted as

$${h}_{{m \_ a}_{i}}^{t} ,$$

and obtaining the fusion hidden layer information of the first region i denoted as h_mᵢ^{t}:

$${h\_m}_{i}^{t} + {w}_{i} * {h}_{{m \_ t}_{i}}^{t} + {w}_{a} * {h}_{{m \_ a}_{i}}^{t} .$$

wᵢ represents a macro weight, wₐ represents a micro weight, and both of wᵢ and wₐ are parameters obtained by automatic updating during training iterations of the infectious disease prediction model.

In an embodiment, at the step 203, performing a time sequence calculation of the fusion hidden layer information to obtain time sequence hidden layer information of the plurality of first regions further includes: acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

Alternatively, when the current period is an initial period, the fusion hidden layer information at the current period and a randomly generated random number may be input into the recurrent neural network to obtain the timing hidden layer information of the plurality of first regions at the current period.

Specifically, based on the time sequence hidden layer information of the first region i in the previous period denoted as hᵢ^{t-1} and the fusion hidden layer information of the first region i denoted as h_mᵢ^{t}, the time sequence hidden layer information of the first region i denoted as hᵢ^{t} at the current period may be obtained by a following formula:

$${h}_{i}^{t} = GRU ( {h}_{{m}_{i}}^{t} , {h}_{i}^{t - 1} ) ,$$

GRU represents the recurrent neural network.

In the present embodiment, the fusion hidden layer information may be input into the recurrent neural network to realize spatial-temporal deep learning of macro-micrograph information and obtain the time sequence hidden layer information of the first region.

In an embodiment, the first result may include a first prediction number of infected persons and a first prediction number of recovered persons, the second result may include a second prediction number of infected persons and a second prediction number of recovered persons, and the second prediction network may include a parameter prediction network and an infection prediction network. At the step 203, inputting the time sequence hidden layer information into the first prediction network and the second prediction network to obtain the corresponding first result and the corresponding second result respectively may further include:
inputting the time sequence hidden layer information into the first prediction network, and outputting the first prediction number of infected persons and the first prediction number of recovered persons in the plurality of first regions; and
inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

Specifically, the time sequence hidden layer information hᵢ^{t} may be input into the first prediction network denoted as MLP_1, and the first prediction number of infected persons denoted as I′ and the first prediction number of recovered persons denoted as R′ of the first region i may be output by a formula: I′, R′=MLP_1(hᵢ^{t}).

The time sequence hidden layer information hᵢ^{t} may be input into the parameter prediction network denoted as MLP_2, and the first prediction parameter denoted as β and the second prediction parameter denoted as γ of the first region i may be output by a formula: β, γ=sigmoid (MLP_2(hᵢ^{t})). The first prediction parameter β and the second prediction parameter γ may be input into the infection prediction network denoted as SIR, and the second prediction number of infected persons denoted as I″ and the second prediction number of recovered persons R″ of the first region i may be output by a formula: I″, R″=SIR(β,γ).

In an embodiment, at the step 204, performing the fusion calculation of the first result and the second result to obtain the infectious disease prediction result of the plurality of first regions may further include:
performing a fusion calculation of the first prediction number of infected persons and the second prediction number of infected persons to obtain a prediction result of the number of infected persons in the plurality of first regions; and
performing a fusion calculation of the first prediction number of recovered persons and the second prediction number of recovered persons to obtain a prediction result of the number of recovered persons in the plurality of first regions.

Specifically, the fusion calculation of the first prediction number of infected persons I′ and the second prediction number of infected persons I″ may be performed to obtain the prediction result of the number of infected persons denoted as/in the first region i by a formula: I=p*I′+q*I″. The fusion calculation of the first prediction number of recovered persons R′ and the second prediction number of recovered persons R″ may be performed to obtain the prediction result of the number of recovered persons denoted as R in the first region i by a formula: R=p*R′+q*R″. p and q are weight parameters and satisfy a formula: p+q=1.

The infectious disease prediction model may be continuously trained during execution of the step 201 to the step 204, and a loss value of the infectious disease prediction model denoted as loss may be calculated by a MSE (mean-square error) method:

$$loss = p * ( MSE ( I , {I}^{′} ) + MSE ( R , {R}^{′} ) ) + q * ( MSE ( I , {I}^{″} ) + MSE ( R , {R}^{″} ) ) .$$

In an example embodiment, an infectious disease infection prediction method based on macro-micrograph fusion is provided. A first region may represent a province, a second region may represent a city of the province, and the method may include following steps 1 to 6:

Step 1 may include: within the set period t, taking a plurality of provinces as first nodes, while utilizing the number of infected persons Sᵣₑₘ_{_{i,j}}^{t} flowing from a province i to a province j, the total number of infected persons Σ_{k∈N(i)}Sᵣₑₘ_{_{i,k}}^{t} flowing out from the province i, the historical number observeᵢ^{t} of infected persons of the province i, the historical number observeⱼ^{t} of infected persons of the province j, and a geographic index ∈ between the province i and the province j, to obtain a first connecting edge probability e_s_{i,j}^{t}, between the first nodes:

$${e\_s}_{i , j}^{t} = a * \frac{{s}_{{rem}_{i , j}^{t}}}{{\sum }_{k ∈ N ( i )} {s}_{{rem}_{i , k}^{t}}} + b * simil ( {observe}_{i}^{t} , {observe}_{j}^{t} ) + ∈ ,$$

connecting two first nodes between which the first connecting edge probability e_s_{i,j}^{t} is greater than a set threshold to obtain a first edge, and generating a macrograph of the plurality of provinces denoted as g_md^{t}=(Vₛ^{t}, Eₛ^{t}). Vₛ^{t} represents the first nodes corresponding to the plurality of provinces, and Eₛ^{t} represents a combination of first connecting edges of the provinces. A total population denoted as Pₛ_{ᵢ}, a population density denoted as Dₛ_{ᵢ}, the number of infected persons denoted as Iₛ^{t}, and the number of recovered persons denoted as Rₛ^{t} in each of the plurality of provinces within the set period t may be taken as node features of a corresponding first node Vₛ^{t}.

Step 2 may include: within the same set period t, taking a plurality of cities of a province as second nodes, calculating a second connecting edge probability between the second nodes denoted as e_c_{m,n}^{t} by the number of infected persons denoted as Cᵣₑₘ_{_{m,n}}^{t} flowing from a city denoted as m to another city denoted as n, the total number of infected persons denoted as Σ_{K∈N(m)}Sᵣₑₘ_{_{m,k}}^{t} flowing out from the city m, and a geographic index denoted as ∈′ between the city m and the another city n:

$${e\_c}_{m , n}^{t} = {a}^{′} * \frac{{c}_{{rem}_{m , n}}^{t}}{{\sum }_{k ∈ N ( m )} {c}_{{rem}_{m , k}}^{t}} + {∈}^{′} ,$$

connecting two second nodes between which the second connecting edge probability e_c_{m,n}^{t} is greater than a set threshold to obtain a second edge, and generating a micrograph of the corresponding city of the plurality of provinces denoted as g_mi^{t}=(V_{c}^{t}, E_{c}^{t}). V_{c}^{t} represents the second nodes corresponding to the plurality of cities, and E_{c}^{t} represents a combination of the second connecting edges of the second nodes. A total population denoted as P_{c}_{ᵢ}, a population density denoted as D_{c}_{ᵢ}, and the number of hospitals denoted as H_{c}_{ᵢ} in each of the plurality of cities may be taken as the node features of a corresponding second node V_{c}^{t}.

Step 3 may include inputting the macrograph g_ma^{t}=(Vₛ^{t}, Eₛ^{t}) into a first graph convolutional neural network to obtain a first hidden layer vector denoted as h_{m_i}_{ᵢ}^{t}, and inputting the micrograph g_mi^{t}=(V_{c}^{t}, E_{c}^{t}) into a second graph convolutional neural network to obtain a second hidden layer vector h_{m_a}_{ᵢ}^{t}, performing a fusion calculation of the first hidden layer vector denoted as h_{m_i}_{ᵢ}^{t}, and the second hidden layer vector denoted as h_{m_a}_{ᵢ}^{t}, and obtaining a fusion hidden layer information h_mᵢ^{t} of the province i:

$${h\_m}_{i}^{t} = {w}_{i} * {h}_{{m \_ i}_{i}}^{t} + {w}_{a} * {h}_{{m \_ a}_{i}}^{t} .$$

Step 4 may include obtaining time sequence hidden layer information hᵢ^{t} of the province i at a current period according to the time sequence hidden layer information hᵢ^{t-1} of the province i at a previous period and the fusion hidden layer information h_mᵢ^{t} of the province i by a formular: hᵢ^{t}=GRU(hₘ_{ᵢ}^{t}, hᵢ^{t-1}).

Step 5 may include inputting the time sequence hidden layer information hᵢ^{t} into a first prediction network MLP_1, and outputting the first prediction number of infected persons I′ and the first prediction number of recovered persons R′ of the province i by a formula: I′, R′=MLP_1(hᵢ^{t}); inputting the time sequence hidden layer information hᵢ^{t} of the province i into a parameter prediction network MLP_2, and outputting a first prediction parameter β and a second prediction parameter γ of the province i by a formula: β, γ=sigmoid(MLP_2 (hᵢ^{t})); and inputting the first prediction parameter β and the second prediction parameter γ into an infection prediction network SIR, and outputting the second prediction number of infected persons I″ and the second prediction number of recovered persons R″ of the province i by a formula: I″, R″=SIR(β, γ).

Step 6 may include perform a fusion calculation of the first prediction number of infected persons I′ and the second prediction number of infected persons I′ to obtain a prediction result I of the number of infected persons of the province i by a formula: I=p*I′+q*I″, and performing a fusion calculation of the first prediction number of recovered persons R′ and the second prediction number of recovered persons R″ to obtain the prediction result of the number of recovered persons R in the province i by a formula: R=p*R′+q*R″. p and q are weight parameters and satisfy a formula: p+q=1.

It should be understood that although the individual steps in the flowcharts involved in the embodiments as described above are shown sequentially as indicated by the arrows, these steps are not necessarily executed sequentially in the order indicated by the arrows. Unless expressly stated herein, there is no strict order limitation on the execution of these steps, and these steps may be executed in other orders. Moreover, at least a portion of the steps in the flowchart involved in the embodiments as described above may include multiple steps or multiple phases, which are not necessarily executed to completion at the same moment but may be executed at different moments, and the order in which these steps or phases are executed is not necessarily sequential, but may be executed in turn or alternatively with other steps or at least a portion of steps or phases in other steps.

Based on the same inventive concept, an infectious disease infection prediction apparatus based on macro-micrograph fusion is provided in an embodiment of the present disclosure for realizing the infectious disease infection prediction method based on macro-micrograph fusion. The solution of solving a problem by the apparatus may be similar to the solution documented in the above-described method, so specific limitations in one or more embodiments of the infectious disease infection prediction apparatus based on macro-micrograph fusion provided below can be referred to the limitations of the infectious disease infection prediction method based on macro-micrograph fusion, which are not repeated herein.

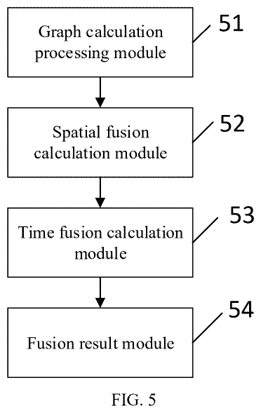

In an embodiment, referring to FIG. 5, an infectious disease infection prediction apparatus based on macro-micrograph fusion is provided, including: a graph calculation processing module 51, a spatial fusion calculation module 52, a time fusion calculation module 53, and a fusion result module 54.

The graph calculation processing module 51 is configured for acquiring macro infection data of a plurality of first regions within a set period to generate macrographs, and acquiring micro infection data of a plurality of second regions within the same set period to generate micrographs corresponding to the plurality of first regions; each of the plurality of first regions includes the plurality of second regions.

The spatial fusion calculation module 52 is configured for inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions.

The time fusion calculation module 53 is configured for performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively.

The fusion result module 54 is configured for performing a fusion calculation of the first result and the second result to obtain an infectious disease prediction result of the plurality of first regions.

In an embodiment, the macro infection data may include macro personnel data and macro geographic data of the plurality of first regions, and the graph calculation processing module 51 is further configured for taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs. The macro personnel data may include: a total population, a population density, the number of infected persons, and the number of recovered persons in each of the plurality of first regions within the set period.

In an embodiment, the graph calculation processing module 51 is further configured for determining a first connecting edge probability between the first nodes based on the macro geographic data of the plurality of first regions, connecting two first nodes between which the first connecting edge probability is greater than a set threshold value, and obtaining the first connecting edges. The macro geographic data may include: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

In an embodiment, the micro infection data comprises micro personnel data and micro geographic data of the plurality of second regions, and the graph calculation processing module 51 is further configured for taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph. The micro personnel data may include: a total population, a population density, and the number of hospitals in each of the plurality of second regions within the set period.

In an embodiment, the graph calculation processing module 51 is further configured for determining a second connecting edge probability between the second nodes based on the micro geographic data of the plurality of second regions, connecting two second nodes between which the second connecting edge probability is greater than a set threshold, and obtaining the second connecting edges. The micro geographic data may include: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

In an embodiment, the time fusion calculation module 53 is further configured for acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

In an embodiment, the first result includes a first prediction number of infected persons and a first prediction number of recovered persons, the second result includes a second prediction number of infected persons and a second prediction number of recovered persons, the second prediction network includes a parameter prediction network and an infection prediction network, and the time fusion calculation module 53 is further configured for inputting the time sequence hidden layer information into the first prediction network, and outputting the first prediction number of infected persons and the first prediction number of recovered persons in the plurality of first regions; and inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

In an embodiment, the fusion result module 54 is further configured for performing a fusion calculation of the first prediction number of infected persons and the second prediction number of infected persons to obtain a prediction result of the number of infected persons in the plurality of first regions; and performing a fusion calculation of the first prediction number of recovered persons and the second prediction number of recovered persons to obtain a prediction result of the number of recovered persons in the plurality of first regions.

The various modules in the above infectious disease infection prediction apparatus based on macro-micrograph fusion may be realized in whole or in part by software, hardware and combinations thereof. Each of the above modules may be embedded in or independent of a processor in a computer device in the form of hardware, or may be stored in a memory in the computer device in the form of software so as to be invoked by the processor to perform the operations corresponding to each of the above modules.

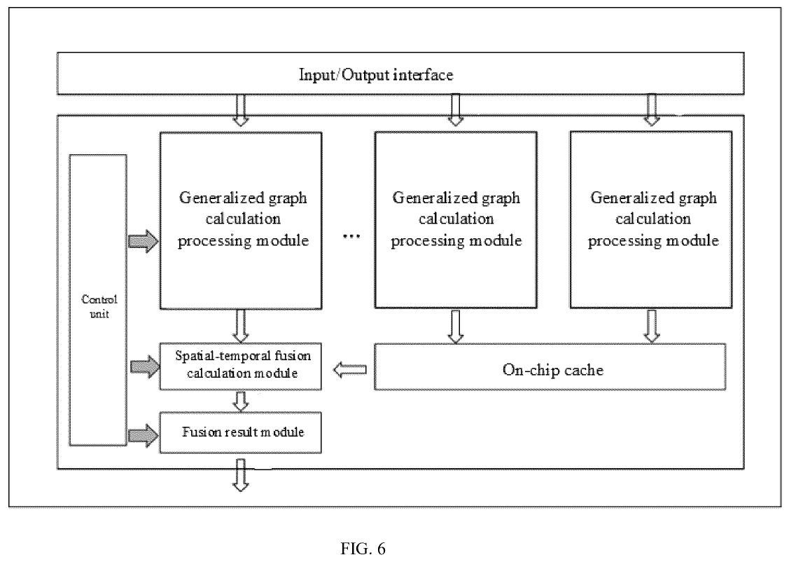

In an embodiment, a FPGA device is provided, an internal structure diagram of which may be shown in FIG. 6. The FPGA device may include a control unit, a generalized graph calculation processing module, a spatial-temporal fusion calculation module, a fusion result module, an on-chip cache, and an input/output interface (I/O). The control unit may be connected to the generalized graph calculation processing module, the spatial-temporal fusion calculation module, the fusion result module, and the Input/Output interface via a system bus. The control unit of the FPGA device is configured to provide computing and control capabilities. The on-chip cache of the FPGA device may include a non-volatile storage medium and an internal memory. The non-volatile storage medium may store an operating system, a computer program, and a database. The input/output interface is configured to exchange information between the generalized graph calculation processing module and an external device. The computer program may be executed by the control unit to implement the infectious disease infection prediction method based on macro-micrograph fusion.

In an embodiment, a computer-readable storage medium is provided on which a computer program is stored, and the computer program is executed by a processor to implement the following steps:
acquiring macro infection data of a plurality of first regions within a set period to generate macrographs, and acquiring micro infection data of a plurality of second regions within the same set period to generate micrographs corresponding to the plurality of first regions; each of the plurality of first regions includes the plurality of second regions;
inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions;
performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively; and
performing a fusion calculation of the first result and the second result to obtain an infectious disease prediction result of the plurality of first regions.

In an embodiment, the macro infection data includes macro personnel data and macro geographic data of the plurality of first regions, and in an embodiment, the computer program is executed by the processor to further implement the following steps: taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs. The macro personnel data may include: a total population, a population density, the number of infected persons, and the number of recovered persons in each of the plurality of first regions within the set period.

In an embodiment, the computer program is executed by the processor to further implement the following steps: determining a first connecting edge probability between the first nodes based on the macro geographic data of the plurality of first regions, connecting two first nodes between which the first connecting edge probability is greater than a set threshold value, and obtaining the first connecting edges. The macro geographic data may include: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

In an embodiment, the micro infection data may include micro personnel data and micro geographic data of the plurality of second regions, the computer program is executed by the processor to further implement the following steps: taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph. The micro personnel data may include: a total population, a population density, and the number of hospitals in each of the plurality of second regions within the set period.

In an embodiment, the computer program is executed by the processor to further implement the following steps: determining a second connecting edge probability between the second nodes based on the micro geographic data of the plurality of second regions, connecting two second nodes between which the second connecting edge probability is greater than a set threshold, and obtaining the second connecting edges. The micro geographic data may include: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

In one embodiment, the computer program is executed by the processor to further implement the following steps: acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

In an embodiment, the first result may include a first prediction number of infected persons and a first prediction number of recovered persons, the second result may include a second prediction number of infected persons and a second prediction number of recovered persons, the second prediction network may include a parameter prediction network and an infection prediction network, and the computer program is executed by the processor to further implement the following steps:
inputting the time sequence hidden layer information into the first prediction network, and outputting the first prediction number of infected persons and the first prediction number of recovered persons in the plurality of first regions; and
inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

In an embodiment, the computer program is executed by the processor to further implement the following steps:
performing a fusion calculation of the first prediction number of infected persons and the second prediction number of infected persons to obtain a prediction result of the number of infected persons in the plurality of first regions; and
performing a fusion calculation of the first prediction number of recovered persons and the second prediction number of recovered persons to obtain a prediction result of the number of recovered persons in the plurality of first regions.

One skilled in the art may understand that realizing all or part of the processes in the methods of the above embodiments is possible to be accomplished by a computer program to instruct relevant hardware, the computer program may be stored in a non-volatile computer-readable storage medium, and the computer program, when executed, may include the processes as in the embodiments of the methods.

The various technical features of the above-described embodiments may be combined in any combination, and all possible combinations of the various technical features of the above-described embodiments have not been described for the sake of conciseness of description. However, as long as there is no contradiction in the combinations of these technical features, they should be considered to be within the scope of the present specification as recorded herein.

The above-described embodiments express only several embodiments of the present disclosure, which are described in a more specific and detailed manner, but are not to be construed as a limitation of the scope of the present disclosure. It should be pointed out that, for one skilled in the art, several deformations and improvements can be made without departing from the conception of the present disclosure, all of which fall within the scope of protection of the present disclosure. Therefore, the scope of protection of this disclosure shall be subject to the attached claims.

What is claimed is:

1. An infectious disease infection prediction method based on macro-micrograph fusion, comprising:
acquiring macro infection data of a plurality of first regions within a set period to generate macrographs, and acquiring micro infection data of a plurality of second regions within the same set period to generate micrographs corresponding to the plurality of first regions, wherein each of the plurality of first regions comprises the plurality of second regions;
inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions;
performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively; and
performing a fusion calculation of the first result and the second result to obtain an infectious disease prediction result of the plurality of first regions.

2. The infectious disease infection prediction method based on macro-micrograph fusion of claim 1, wherein the macro infection data comprises macro personnel data and macro geographic data of the plurality of first regions, and acquiring macro infection data of the plurality of first regions within the set period to generate the macrographs further comprises:
taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs, wherein the macro personnel data comprises: a total population, a population density, the number of infected persons, and the number of recovered persons in each of the plurality of first regions within the set period.

3. The infectious disease infection prediction method based on macro-micrograph fusion of claim 2, wherein determining first connecting edges of the first nodes based on the macro geographic data of the plurality of first regions further comprises:
determining a first connecting edge probability between the first nodes based on the macro geographic data of the plurality of first regions, connecting two first nodes between which the first connecting edge probability is greater than a set threshold value, and obtaining the first connecting edges, wherein the macro geographic data comprises: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

4. The infectious disease infection prediction method based on macro-micrograph fusion of claim 1, wherein the micro infection data comprises micro personnel data and micro geographic data of the plurality of second regions, and acquiring micro infection data of the plurality of second regions within the same set period to generate micrographs corresponding to the plurality of first regions further comprises:
taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph, wherein the micro personnel data comprises: a total population, a population density, and the number of hospitals in each of the plurality of second regions within the set period.

5. The infectious disease infection prediction method based on macro-micrograph fusion of claim 4, wherein determining second connecting edges of the second nodes based on the micro geographic data of the plurality of second regions further comprises:
determining a second connecting edge probability between the second nodes based on the micro geographic data of the plurality of second regions, connecting two second nodes between which the second connecting edge probability is greater than a set threshold, and obtaining the second connecting edges, wherein the micro geographic data comprises: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

6. The infectious disease infection prediction method based on macro-micrograph fusion of claim 1, wherein performing the time sequence calculation of the fusion hidden layer information to obtain time sequence hidden layer information of the plurality of first regions further comprises:
acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

7. The infectious disease infection prediction method based on macro-micrograph fusion of claim 1, wherein the first result comprises a first prediction number of infected persons and a first prediction number of recovered persons, the second result comprises a second prediction number of infected persons and a second prediction number of recovered persons, the second prediction network comprises a parameter prediction network and an infection prediction network, and inputting the time sequence hidden layer information into the first prediction network and the second prediction network to obtain the corresponding first result and the corresponding second result respectively further comprises:
inputting the time sequence hidden layer information into the first prediction network, and outputting the first prediction number of infected persons and the first prediction number of recovered persons in the plurality of first regions; and
inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

8. The infectious disease infection prediction method based on macro-micrograph fusion of claim 7, wherein performing the fusion calculation of the first result and the second result to obtain the infectious disease prediction result of the plurality of first regions further comprises:
performing a fusion calculation of the first prediction number of infected persons and the second prediction number of infected persons to obtain a prediction result of the number of infected persons in the plurality of first regions; and
performing a fusion calculation of the first prediction number of recovered persons and the second prediction number of recovered persons to obtain a prediction result of the number of recovered persons in the plurality of first regions.

9. An infectious disease infection prediction apparatus based on macro-micrograph fusion, comprising a graph calculation processing module, a spatial fusion calculation module, a time fusion calculation module, and a fusion result module;
wherein the graph calculation processing module is configured for acquiring macro infection data of a plurality of first regions within a set period to generate macrographs, and acquiring micro infection data of a plurality of second regions within the same set period to generate micrographs corresponding to the plurality of first regions, wherein each of the plurality of first regions comprises the plurality of second regions;
the spatial fusion calculation module is configured for inputting the macrographs into a first graph convolutional neural network to obtain a first hidden layer vector, inputting the micrographs into a second graph convolutional neural network to obtain a second hidden layer vector, and performing a fusion calculation of the first hidden layer vector and the second hidden layer vector to obtain fusion hidden layer information of the plurality of first regions;
the time fusion calculation module is configured for performing a time sequence calculation of the fusion hidden layer information to obtain time sequence hidden layer information of the plurality of first regions, and inputting the time sequence hidden layer information into a first prediction network and a second prediction network to obtain a corresponding first result and a corresponding second result, respectively; and
the fusion result module is configured for performing a fusion calculation of the first result and the second result to obtain an infectious disease prediction result of the plurality of first regions.

10. A computer-readable storage medium, storing a computer program, wherein the computer program is executed by a processor to implement steps of the method of claim 1.

11. The computer-readable storage medium of claim 10, wherein the macro infection data comprises macro personnel data and macro geographic data of the plurality of first regions, and acquiring macro infection data of the plurality of first regions within the set period to generate the macrographs further comprises:
taking the plurality of first regions as first nodes of the macrographs, acquiring the macro personnel data of the plurality of first regions within the set period as node features of the first nodes, and determining first connecting edges of the first nodes based on the macro geographic data of the plurality of first regions to generate the macrographs, wherein the macro personnel data comprises: a total population, a population density, the number of infected persons, and the number of recovered persons in each of the plurality of first regions within the set period.

12. The computer-readable storage medium of claim 11, wherein determining first connecting edges of the first nodes based on the macro geographic data of the plurality of first regions further comprises:
determining a first connecting edge probability between the first nodes based on the macro geographic data of the plurality of first regions, connecting two first nodes between which the first connecting edge probability is greater than a set threshold value, and obtaining the first connecting edges, wherein the macro geographic data comprises: the number of infected persons flowing from one of the plurality of first regions to another of the plurality of first regions, the total number of infected persons flowing out from the one of the plurality of first regions, the historical number of infected persons in the one of the plurality of first regions, the historical number of infected persons in the another of the plurality of first regions, and a geographic index between the one of the plurality of first regions and the another of the plurality of first regions within the set period.

13. The computer-readable storage medium of claim 10, wherein the micro infection data comprises micro personnel data and micro geographic data of the plurality of second regions, and acquiring micro infection data of the plurality of second regions within the same set period to generate micrographs corresponding to the plurality of first regions further comprises:
taking the plurality of second regions as second nodes of a micrograph, acquiring the micro personnel data of the plurality of second regions as node features of the second nodes, and determining second connecting edges of the second nodes based on the micro geographic data of the plurality of second regions to generate the micrograph, wherein the micro personnel data comprises: a total population, a population density, and the number of hospitals in each of the plurality of second regions within the set period.

14. The computer-readable storage medium of claim 13, wherein determining second connecting edges of the second nodes based on the micro geographic data of the plurality of second regions further comprises:
determining a second connecting edge probability between the second nodes based on the micro geographic data of the plurality of second regions, connecting two second nodes between which the second connecting edge probability is greater than a set threshold, and obtaining the second connecting edges, wherein the micro geographic data comprises: the number of infected persons flowing from one of the plurality of second regions to another of the plurality of second regions, the total number of infected persons flowing out from the one of the plurality of second regions, and a geographic index between the one of the plurality of second regions and the another of the plurality of second regions within the set period.

15. The computer-readable storage medium of claim 10, wherein performing the time sequence calculation of the fusion hidden layer information to obtain time sequence hidden layer information of the plurality of first regions further comprises:
acquiring time sequence hidden layer information of the plurality of first regions at a previous period, inputting the fusion hidden layer information and corresponding time sequence hidden layer information of the plurality of first regions at the previous period into a recurrent neural network, and obtaining the time sequence hidden layer information of the plurality of first regions at a current period.

16. The computer-readable storage medium of claim 10, wherein the first result comprises a first prediction number of infected persons and a first prediction number of recovered persons, the second result comprises a second prediction number of infected persons and a second prediction number of recovered persons, the second prediction network comprises a parameter prediction network and an infection prediction network, and inputting the time sequence hidden layer information into the first prediction network and the second prediction network to obtain the corresponding first result and the corresponding second result respectively further comprises:
inputting the time sequence hidden layer information into the first prediction network, and outputting the first prediction number of infected persons and the first prediction number of recovered persons in the plurality of first regions; and
inputting the time sequence hidden layer information into the parameter prediction network, outputting a first prediction parameter and a second prediction parameter of the plurality of first regions, inputting the first prediction parameter and the second prediction parameter into the infection prediction network, and outputting the second prediction number of infected persons and the second prediction number of recovered persons in the plurality of first regions.

17. The computer-readable storage medium of claim 16, wherein performing the fusion calculation of the first result and the second result to obtain the infectious disease prediction result of the plurality of first regions further comprises:
performing a fusion calculation of the first prediction number of infected persons and the second prediction number of infected persons to obtain a prediction result of the number of infected persons in the plurality of first regions; and
performing a fusion calculation of the first prediction number of recovered persons and the second prediction number of recovered persons to obtain a prediction result of the number of recovered persons in the plurality of first regions.
